(19) United States
(12) Patent Application Publication
(10) Pub. No.: US 2019/0275264 A9
(43) Pub. Date: Sep. 12, 2019

(54) INHALATION DEVICE WITH CONSUMPTION METERING WITHOUT AIRFLOW SENSORS

(71) Applicant: INDOSE INC, Woodland Hills, CA (US)

(72) Inventors: Daniel Freeman, Agoura, CA (US); Ari Freeman, Lafayette, CA (US); Jacqueline Freeman, Lafayette, CA (US)

(21) Appl. No.: 16/114,372

(22) Filed: Aug. 28, 2018

Related U.S. Application Data
(63) Continuation-in-part of application No. 15/244,518, filed on Aug. 23, 2016.
(60) Provisional application No. 62/621,795, filed on Jan. 25, 2018.
(60) Provisional application No. 62/386,614, filed on Dec. 7, 2015.
(60) Provisional application No. 62/386,615, filed on Dec. 7, 2015.
(60) Provisional application No. 62/388,066, filed on Jan. 13, 2016.

(51) Int. Cl. A61M 11/00; A61M 15/00

(52) U.S. Cl. CPC A61M 11/00; A61M 2205/50; A61M 2205/3313; A61M 15/002

(57) ABSTRACT

An inhalation device for inhaling a vaporized substance that includes metering capabilities to inform a user when a particular amount of substance has been consumed. The device includes an inlet having an opening, an outlet, a processor, an atomizer configured to vaporize an unvaporized substance into a vaporized substance. The device further includes a channel positioned between the atomizer and the outlet, wherein the vaporized substance flows downstream from the atomizer to the outlet via the channel, a light signal device, wherein the light signal device emits light, and a light sensor, wherein the light sensor senses the light from the light signal device. In addition, the light signal device and the sensor are positioned in the channel such that the vaporized substance can flow past the sensor and the light signal device, the opening is configured to allow entry of air into the device that flows to the atomizer, the inlet is configured such that the air flows at a substantially constant rate, and the processor, using the substantially constant rate and the data from the light sensor, is configured to meter an amount of vapor consumed by a user.

This application is a continuation-in-part of and claims priority to U.S. patent application Ser. No. 15/244,518, filed on Aug. 23, 2016, which in turn claims priority to U.S. Provisional Patent Application Nos. 62/386,614 and 62/386,615, both of which were filed on Dec. 7, 2015, and 62/388,066, which was filed on Jan. 13, 2016. This application also claims priority to U.S. Provisional Patent Application No. 62/621,795 filed on Jan. 25, 2018. All of these applications are incorporated by reference herein in their entireties.

### BACKGROUND

Inhaling devices such as vaporizers, vaporizing pens, and vaporizing machines are used to vaporize substances such as tobaccos, oils, liquids, medical drugs, and plant herbs. Once vaporized, these substances are then inhaled by consumers. Such inhaling devices have health benefits over traditional smoking methods. But inhaling the vapor can have negative effects on the body depending on the substance, such as nicotine. Inhaling devices have become more popular with consumers, but pose problems.

For example, while vaporizers can be safer than traditional smoking methods, it is difficult to meter the amount of vaporized substance that is being inhaled. So a user of an inhalation device that vaporizes nicotine may actually consume more nicotine than had the user smoked cigarettes or cigars.

There are multiple factors that affect the quantity of drug that is inhaled. These factors include the drug concentration of the vaporized substance, the amount of vapor inhaled, the duration of inhalation, variations between inhalation devices, and variation and inconsistency in the functionality of the device.

Another issue is that the inhaled substances may have different effects on different users depending on various factors. To optimize a user's experience, it is necessary to track the quantity inhaled taken over time and track the resulting effect it has on that user. This can be a tedious and demanding task. Typical users may not keep track of each dose and record the experience.

### SUMMARY

Various aspects and embodiments of inhalation devices are provided in this disclosure. In one aspect, this disclosure describes an inhalation device for inhaling a vaporized substance that includes metering capabilities to inform a user when a particular amount of substance has been consumed. The device includes an inlet having an opening, an outlet, a processor, an atomizer configured to vaporize an unvaporized substance into a vaporized substance. The device further includes a channel positioned between the atomizer and the outlet, wherein the vaporized substance flows downstream from the atomizer to the outlet via the channel, a light signal device, wherein the light signal device emits light, and a light sensor, wherein the light sensor senses the light from the light signal device. In addition, the light signal device and the sensor are positioned in the channel such that the vaporized substance can flow past the sensor and the light signal device, the opening is configured to allow entry of air into the device that flows to the atomizer, the inlet is configured such that the air flows at a substantially constant rate, and the processor, using the substantially constant rate and the data from the light sensor, is configured to meter an amount of vapor consumed by a user.

In another aspect, the disclosure provides an inhalation device for inhaling a vaporized substance including an inlet, an outlet, a processor, an atomizer positioned between the inlet and the outlet and configured to vaporize an unvaporized substance into a vaporized substance, the vaporized substance flows downstream from the atomizer to the outlet via the channel. The device further includes a light signal device, wherein the light signal device emits light, a light sensor, wherein the light sensor senses the light from the light signal device, the light signal device and the sensor are positioned in the channel such that the vaporized substance can flow past the sensor and the light signal device, the processor is configured to determine a vapor concentration using data from the sensor, the processor is configured to determine an increase or decrease in vapor concentration, and the processor, using the vapor concentration and the increase or decrease in vapor concentration, is configured to meter an amount of vapor consumed by a user.

In another aspect, the disclosure provides an inhalation device for inhaling a vaporized substance that includes an inlet having an opening, an outlet, a processor, an atomizer configured to vaporize an unvaporized substance into a vaporized substance, a channel positioned between the atomizer and the outlet. The device further includes that the vaporized substance flows downstream from the atomizer to the outlet via the channel, a first light signal device, wherein the first light signal device emits light, a first light sensor, the light sensor senses the light from the first light signal device, a second light signal device, a second light sensor, the second light sensor senses the light from the second light signal device, the first light signal device and first light sensor are positioned upstream of the second light signal device and the second light sensor; and the processor, using data from the first and second light sensors, is configured to meter an amount of vapor consumed by a user.

### DESCRIPTION OF THE DRAWINGS

FIG. 1 is a diagram of an inhalation device, according to an embodiment of this disclosure.

FIG. 2 is another diagram of an inhalation device, according to an embodiment of this disclosure.

FIG. 3 is another diagram of an inhalation device, according to an embodiment of this disclosure.

FIG. 4 is another diagram of an inhalation device, according to an embodiment of this disclosure.

FIG. 5 is another diagram of an inhalation device, according to an embodiment of this disclosure.

FIG. 6 is a diagram of a portion of an inhalation device, according to an embodiment of this disclosure.

FIG. 7 is a diagram of a portion of an inhalation device, according to an embodiment of this disclosure.

FIG. 8 another diagram of an inhalation device, according to an embodiment of this disclosure.

FIG. 9 is a graph illustrating performance of an inhalation device according to an embodiment of this disclosure.

FIG. 10 is a diagram of a portion of an inhalation device, according to an embodiment of this disclosure.

FIG. 11 is a graph illustrating performance of an inhalation device according to an embodiment of this disclosure; and

FIG. 12 is a chart showing optosensor output change with increasing vapor intensity.

### DETAILED DESCRIPTION

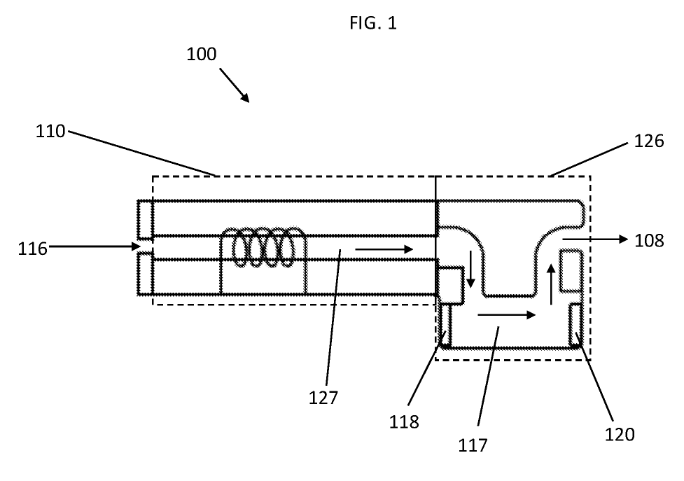

The embodiments described herein disclose an inhalation device that meter consumption without the need for a separate airflow sensor. For example, FIG. 1 illustrates an inhalation device 100 according to an embodiment of this disclosure. More specifically, inhalation device 100 includes an inlet 116, an atomizer 110, a vapor sensing unit 126 and an outlet 108. The atomizer 110 includes a channel 127 and the vapor sensing unit 126 includes a signal 118, a sensor 120, and a channel 117. The atomizer 110 produces vapor that a user inhales through the outlet 108. The vapor will flow in the channel 127 of the atomizer 110 and through channel 117 of the vapor sensing unit 126 before flowing through the outlet 108. The signal 118 and sensor 120 are positioned for sensing concentration of the vapor that flows in a channel 117. The signal 118 and sensor 120 are positioned on ends of the channel 117.

The sensor 120 senses the vapor amount. For example, the sensor 120 can sense the concentration of vapor. The sensor 120 senses the intensity of the signal emitted by the signal 118. If the sensor 120 senses a high signal output, this indicates that the amount of vapor is low, and the vapor/air mixture is dominated by air. Likewise, if the sensor 120 senses a low signal output, this indicates that the vapor/air mixture is dominated by vapor.

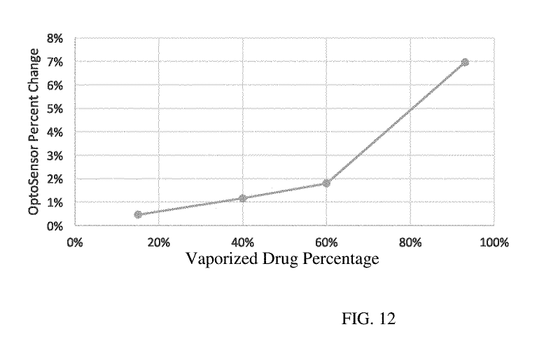

Data from the sensor 120 can assist the device 100 in providing information about vapor concentration to the user. For example, if the sensor senses a 5% drop in intensity from the signal 118, that could correlate to a mixture of vapor/air that is 60% vapor. The chart of FIG. 12 graphs the value percent drop in an optocell (i.e., a device that senses the intensity of light) versus the percentage of vaporized drug in a mixture of vapor and air.

FIG. 12 shows the correlation between vapor concentration and the readings from an optocell. Knowing the relative concentration of the vapor can assist the device 100 in providing additional information to the user. For example, if a user inhales using the device 100 and the sensor 120 senses a high output, this may indicate that the concentration is less than expected. The device 100 could include an additional indicator to inform the user that the device 100 is not producing the expected amount of vapor. The sensor 120 can be any suitable sensor that senses light including without limitation, a photosensor, photodetector, optocell, optoresistor, optotransistor, optodiode, and/or solar cell. The signal 118 can be any suitable device that produces light, such as an LED. The signal could also emit ultraviolet light. In other words, the signal 118 can produce a wide range of wavelengths of light and the sensor 108 detects those wavelengths of light. The inhalation device 100 can optionally use filters in order to target a specific wavelength of light to optimally detect vapor intensity.

In addition, the signal 118 can also be tuned to particular wavelengths or a plurality of wavelengths to detect specific types of molecules and quantities of these molecules that are present in the passing vapor. This would allow identification and quantification of drugs in vaporized form. This technology can be fitted in a small and limited space such as a compact inhalation device. The vapor itself can remain in its current unaltered state during analysis. The technology allows for real-time analysis as it is being inhaled by the user. Several wavelengths of light may be used concurrently.

While the signal 118 and the sensor 120 are able to determine vapor concentration, determining the volume of the vapor is needed to ultimately meter the quantity of drug consumed by a user. Traditionally a sensor for measurement of volume of flow would be needed to measure the flow rate of the vapor. This data would be combined with the vapor concentration to derive a mass flow rate of vapor and/or substance. A person having ordinary skill in the art would understand that an atomizer produces vapor at varying degrees, and a user may inhale at varying intensities leading to a variable flow rate through a typical inhalation device. Having an airflow sensor to sense this variable data would typically be required. However, in the inhalation device 100, the airflow rate is restricted to a substantially set (limited) rate. As a result, there is no need to measure the air flow with a separate sensor. The mass flow rate can be derived based on the known flow rate of the device 100 and the vapor concentration.

Specifically, and still referring to FIG. 1, limiting the flow rate to a substantially particular rate can be achieved through physical design of the air and/or vapor flow pathway. For instance, in FIG. 1, the inlet 116 can be of a specific diameter that may constrain the air flow to a substantially set/limited flow rate. As shown in FIG. 1, the diameter of the inlet 116 is reduced to a small magnitude, for example around 1 mm in diameter to essentially obtain a constant flow of air into the inhalation device from the inlet 116.

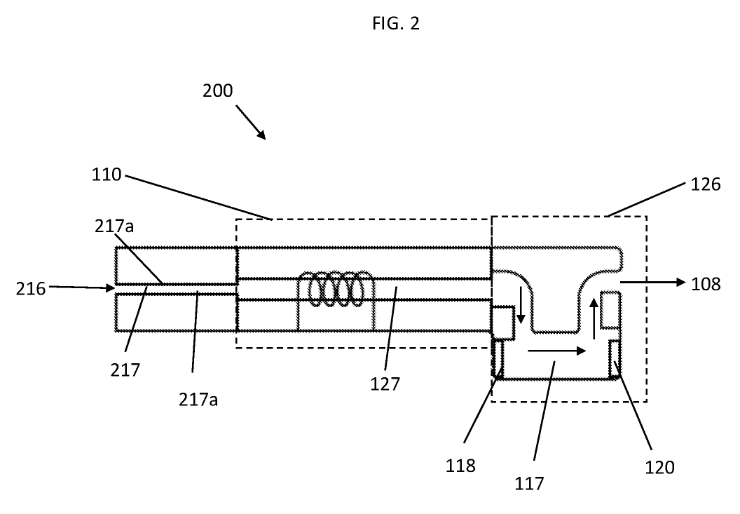

Alternatively, the inlet of an inhalation device can also be elongated to maintain a constant air flow. This is illustrated in FIG. 2, which shows inhalation device 200 according to another embodiment of the disclosure. FIG. 2 includes the elements of FIG. 1 with the exception being that an inlet 216 of FIG. 2 is longer than the inlet 116 of FIG. 1, and comprises a channel 217. The channel 217 is used to control and limit the air flow rate through this channel by surface tension and friction between the air and the sidewalls 217a of the channel 217. In an embodiment, the diameter of the hole maybe around 1 mm and the length approximately from 5-10 mm.

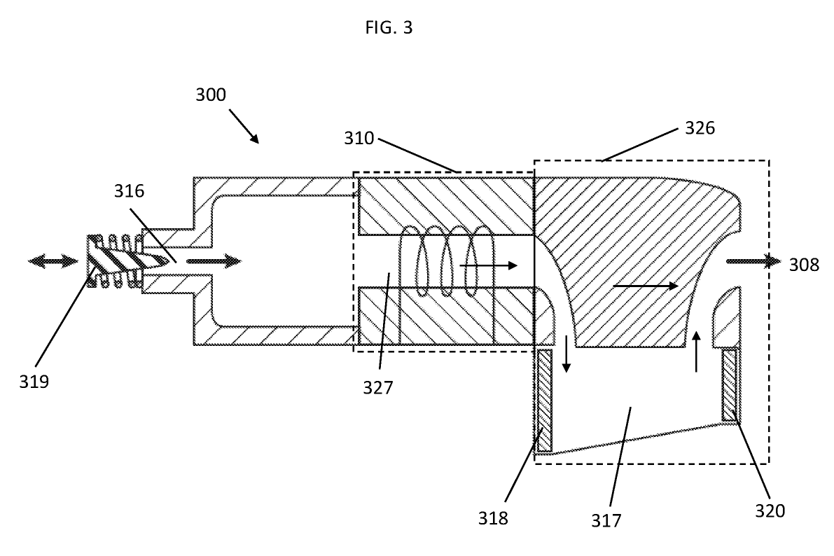

FIG. 3 illustrates an inhalation device 300 according to another embodiment of this disclosure. More specifically, inhalation device 300 includes an inlet 316, an atomizer 310, a vapor sensing unit 326 and an outlet 308. The atomizer 310 includes a channel 327 and the vapor sensing unit 326 includes a signal 318, a sensor 320, and a channel 317. The atomizer 310 produces vapor that a user inhales through the outlet 308. The vapor will flow in the channel 327 of the atomizer 310 and through channel 317 of the vapor sensing unit 326 before flowing through the outlet 308. The signal 318 and sensor 320 are positioned for sensing concentration of the vapor that flows in a channel 317. The signal 318 and sensor 320 are positioned on ends of the channel 317.

FIG. 3 also includes a plunger 319. The plunger 319 can move in an axial direction into and out of the inlet 316 and may be shaped like a coned needle that penetrates the inlet 319. The plunger 319 may be biased away from the inlet 316 such that the higher the air flow rate (i.e., the more intense a user inhales), the more the plunger 319 gets “sucked in” to the hole and restricts the air flow rate. The plunger 319 can thus be used to restrict the air flow rate to a substantially set flow rate regardless of the intensity with which a user inhales.

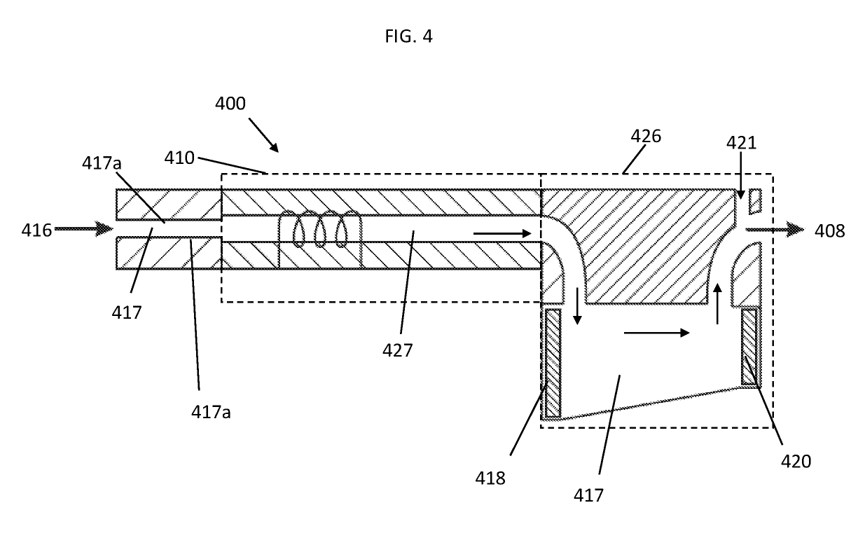

FIG. 4 illustrates an inhalation device 400 according to another embodiment of the disclosure. More specifically, inhalation device 400 includes an inlet 416, an atomizer 410, a vapor sensing unit 426 and an outlet 408. The atomizer 410 includes a channel 427 and the vapor sensing unit 426 includes a signal 418, a sensor 420, and a channel 417. The atomizer 410 produces vapor that a user inhales through the outlet 408. The vapor will flow in the channel 427 of the atomizer 410 and through channel 417 of the vapor sensing unit 426 before flowing through the outlet 408. The signal 418 and sensor 420 are positioned for sensing concentration of the vapor that flows in a channel 417. The signal 418 and sensor 420 are positioned on ends of the channel 417. The inlet 416 of the device 400 is elongated and comprises a channel 417. The channel 417 is used to control and limit the air flow rate through this channel by surface tension and friction between the air and the sidewalls 417a of the channel 417.

To allow a user to inhale faster, while controlling the airflow rate in the atomizer 410 and/or the vapor sensing unit 426, the device 400 includes a second air inlet 421 that is separated from the airflow of the atomizer 410 and the vapor sensing unit 426. This allows a known airflow rate in the channel 417, while allowing a user the freedom to experience a varying airflow rate.

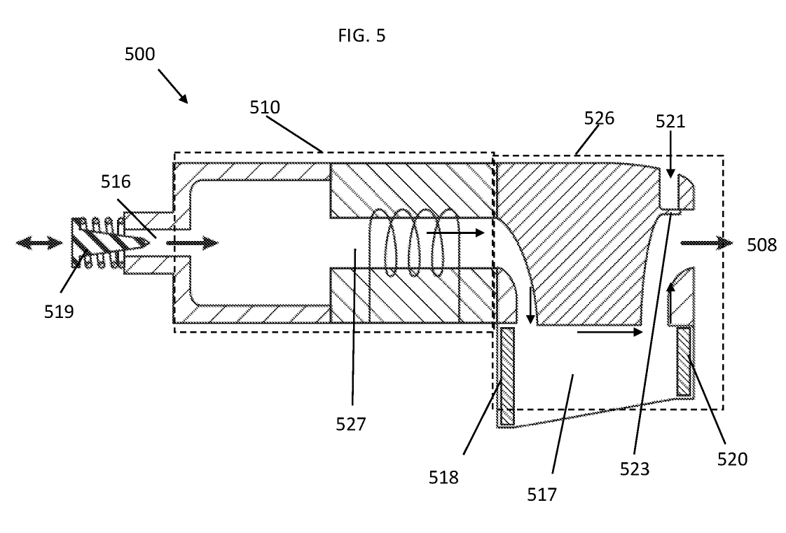

FIG. 5 illustrates an inhalation device 500 according to another embodiment of the disclosure. More specifically, inhalation device 500 includes an inlet 516, an atomizer 510, a vapor sensing unit 526 and an outlet 508. The atomizer 510 includes a channel 527 and the vapor sensing unit 526 includes a signal 518, a sensor 520, and a channel 517. The atomizer 510 produces vapor that a user inhales through the outlet 508. The vapor will flow in the channel 527 of the atomizer 510 and through channel 517 of the vapor sensing unit 526 before flowing through the outlet 508. The signal 518 and sensor 520 are positioned for sensing concentration of the vapor that flows in a channel 517. The signal 518 and sensor 520 are positioned on ends of the channel 517. The device 500 also includes a plunger 519 that operates as describe with respect to plunger 319. In addition, the device includes a second inlet 521 having a valve 523. In this embodiment, the valve 523 of the second inlet 521 is biased in the closed position and opens after a certain airflow rate threshold is reached inside the device 500. This will ensure that air will first enter via the inlet 516 to the atomizer 510 and vapor sensing unit 526. Only after a certain airflow is reached, will the second inlet 521 be open. A threshold could be set around 20 ml/sec, so when a faster rate is presented by the user, it will open the second hole and air will come in from there. This results in a consistent airflow rate in the first hole and along the atomizer.

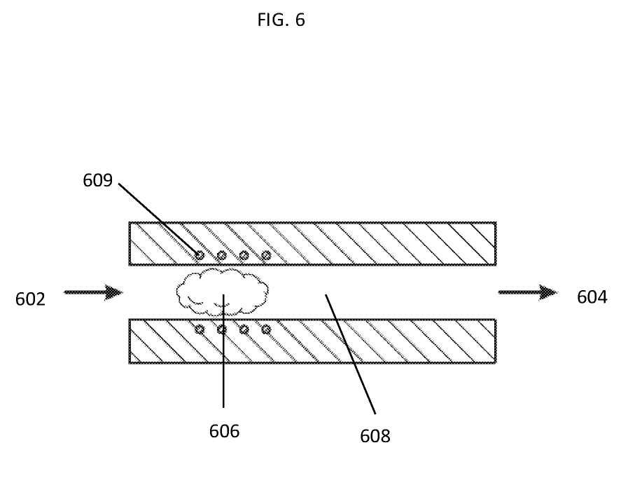

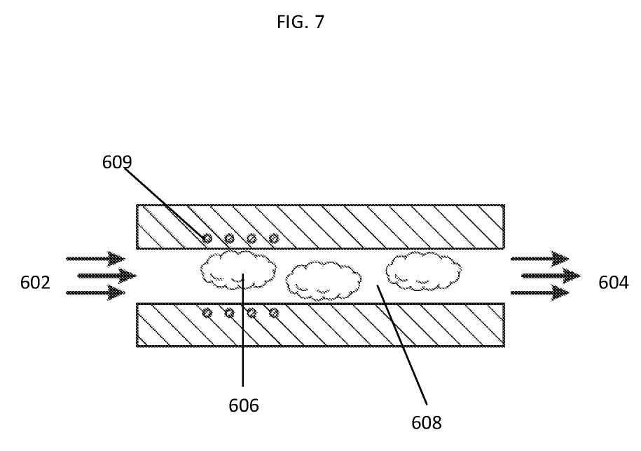

In another aspect of the present disclosure, controlling the airflow rate of an inhalation device can be derived without substantially restricting the airflow rate and without a sensor for measuring data relating to air flow rate. This embodiment includes that variations in the vapor production essentially match variations in the airflow rate. So if the airflow rate increases by 50%, then the vapor production rate needs to increase by approximately 50%. In this embodiment, the vapor sensor (as described in various embodiments herein) will identify these increases in vapor density and account accordingly. Implementation of this embodiment can be achieved by design considerations to where the vapor is being product, e.g., the atomizer of embodiments described herein. For example, the specific area of vaporization (where the liquid vaporizes) can be designed in such a way that this space may become saturated with vapor at a certain point. FIG. 6 shows the vapor saturation 606 created by heating element 609 in the vaporization area 608. As the air flows past this area (illustrated by the arrow from the inlet 602 through to the outlet 604), it will carry the vapor and provide un-saturated air to that space, which will in-turn get saturated, and so on. The slower the air moves, the less vapor is created per unit time. The faster the air moves, the more vapor is produced per unit time. FIG. 7 shows air flowing more quickly through the vaporization area and moving the vapor with it and allowing more vapor to be produced. In the embodiments shown in FIGS. 6 and 7, the inhalation rate is not known; however, the increases and decreases in vapor density are measured by the vapor sensing unit as described herein and are accounted for by the microprocessor. Considering that a human has a limited range of inhalation rates, the embodiments described in FIGS. 6 and 7 can provide substantially accurate results.

In another embodiment, substantially accurate results as to determining airflow rate for an inhalation device may be derived without substantially restricting the airflow rate and without a sensor for measuring data relating to air flow rate and without a vapor sensor. This embodiment includes that the vapor production needs to be consistent with respect to time. For example, if the vaporization unit produced a set amount of vapor per second, say 1 mg/second, then the total amount of drug can be calculated based on duration of puff alone. In such a setup, the production of vapor would need to be independent of uncontrolled variables such as air flow rates.

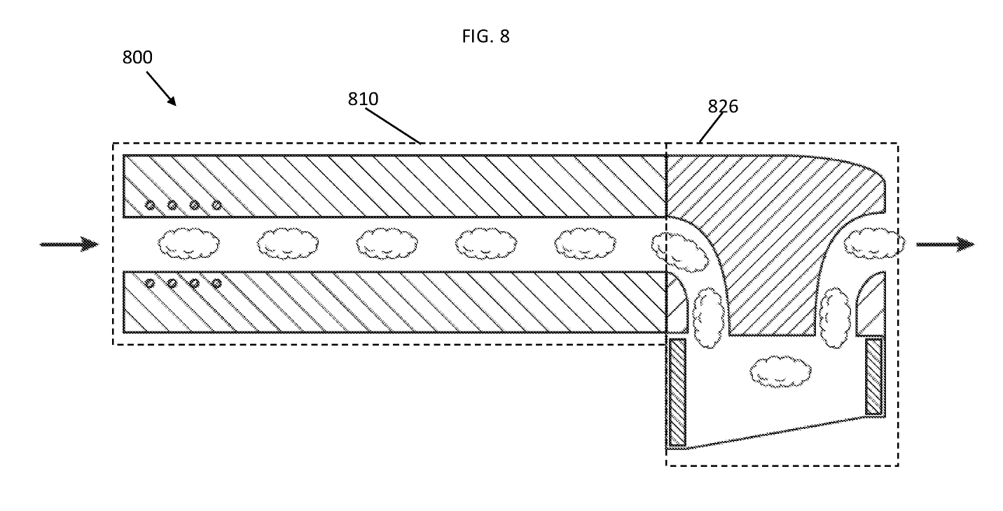

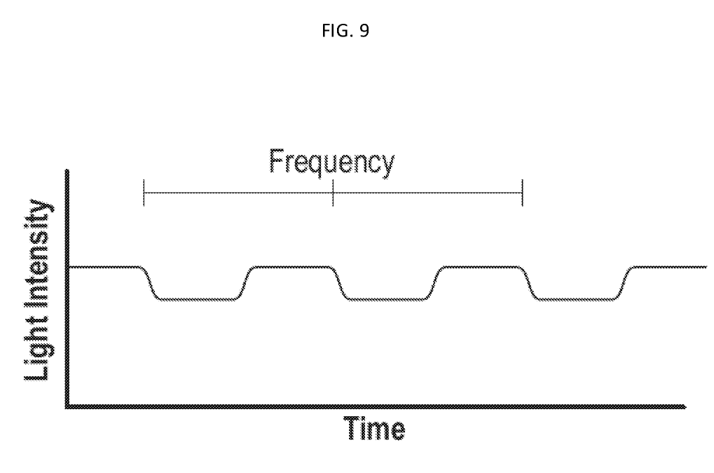

Yet another embodiment provides a way to derive the flow rate of the vapor by use of the vapor sensing unit as described herein. The vapor sensing unit may be setup in a way as to provide a pattern (or rhythm) to the vapor production. For example, the production of the vapor may be pulsed (on-off) at a known certain frequency, as shown in FIG. 8. FIG. 8 shows an inhalation device 800 that includes an atomizer 810 and a vapor sensing unit 826 as described in various embodiments herein. The vapor sensing unit would identify these pulses in vapor production as increases and decreases in vapor density. By comparing the frequency of the identified pulses to the known frequency of vapor production, the flow rate of the vapor can be determined as shown in FIG. 9. This may be determined by calculation or by experimentation. In parallel, the density of the vapor can be determined by the intensity of the light for each pulse. This method would not be limited to on-off pulses. For example, a sine wave pattern may be chosen.

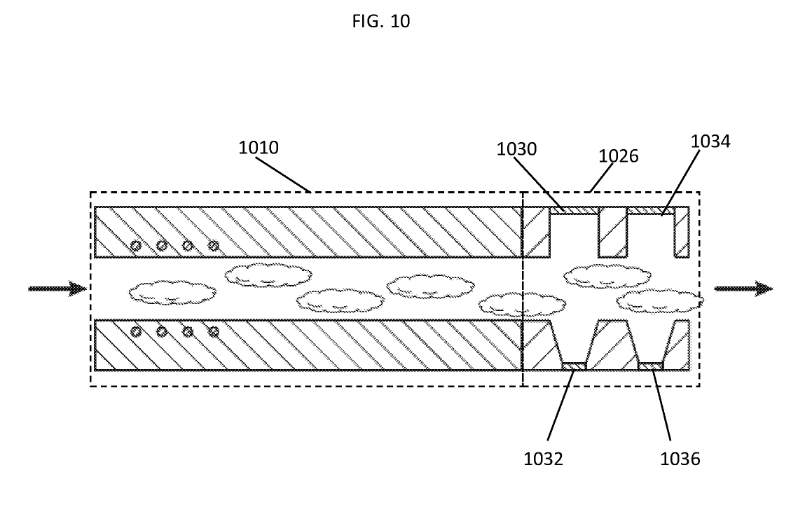

In another embodiment the vapor sensor is used to identify vapor flow rate by having a duel vapor sensor setup. This is illustrated in FIG. 10, which shows a portion of an inhalation device having an atomizer 1010 and a vapor sensing unit 1026. The vapor sensing unit 1026 has a first light sensor 1030, a first light source 1032, a second light sensor 1034, and a second light source 1036. As shown in FIG. 10, vapor would flows past the two vapor sensors 1030 and 1034 that are positioned in such a way that the vapor passes by the first sensor 1030 before passing by the second sensor 1034. Both sensors 1030 and 1034 would record the passing vapor intensity profile, and the detailed fluctuations that naturally occur during in vapor.

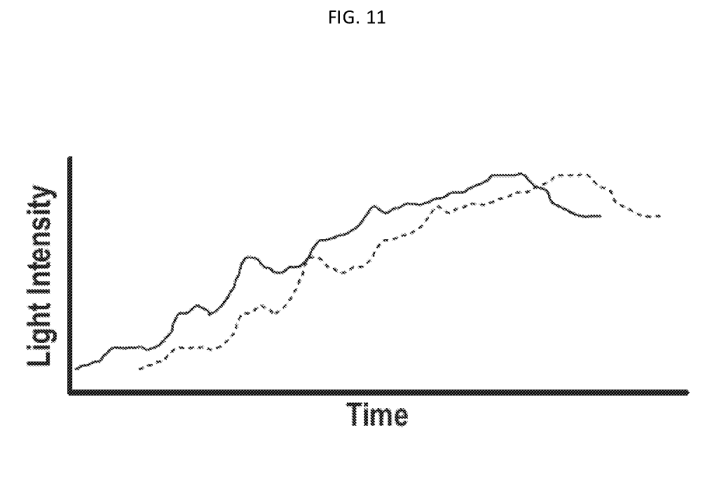

The two sensors 1030 and 1034 will record essentially the same profiles and details, however at different times due to their different positions in the pathway. The microprocessor will analysis the two profiles, find matching reference points in both, and calculate the time offset. Based on the time offset and physical distance between these sensors, the flow rate may be calculated. FIG. 11 for example, shows the two profiles over time. The first sensor 1030 is the solid line and the second sensor 1034 is the broken line.

While embodiments have been illustrated and described herein, it is appreciated that various substitutions and changes in the described embodiments may be made by those skilled in the art without departing from the spirit of this disclosure. The embodiments described herein are for illustration and not intended to limit the scope of this disclosure.

1. An inhalation device for inhaling a vaporized substance comprising: an inlet having an opening;
an outlet;
a processor;
an atomizer configured to vaporize an unvaporized substance into a vaporized substance; a channel positioned between the atomizer and the outlet, wherein the vaporized substance flows downstream from the atomizer to the outlet via the channel;
a light signal device, wherein the light signal device emits light;
a light sensor, wherein the light sensor senses the light from the light signal device;
wherein the light signal device and the sensor are positioned in the channel such that the vaporized substance can flow past the sensor and the light signal device;
wherein the opening is configured to allow entry of air into the device that flows to the atomizer, wherein the inlet is configured such that the air flows at a substantially constant rate; and
wherein the processor, using the substantially constant rate and the data from the light sensor, is configured to meter an amount of vapor consumed by a user.

2. The inhalation device of claim 1 wherein the sensor and the light signal device are positioned across from each other in the channel such that the vaporized substance can flow between the sensor and the light signal device.

3. The inhalation device of claim 1 wherein the light sensor and the light signal device are positioned next to each other.

4. The inhalation device of claim 1 wherein the light sensor and the light signal device are positioned at an angle in the channel of the inhalation device.

5. The inhalation device of claim 1 wherein the inlet comprises a channel having at least two sidewalls, wherein the flow rate through the channel is limited by surface tension and friction between the air and the sidewalls.

6. The inhalation device of claim 1 further comprising a second inlet, wherein the second inlet provides airflow into the inhalation device.

7. The inhalation device of claim 6 wherein the second inlet includes a valve.

8. The inhalation device of claim 1 wherein the device further includes a plunger that is positioned at the inlet and is configured to move in an axial direction to limit airflow into the device.

9. The inhalation device of claim 1 wherein the light signal device is tuned to output a particular wavelength of light.

10. The inhalation device of claim 9, wherein the light sensor is configured using a filter to detect the particular wavelength of light.

11. The inhalation device of claim 1 wherein the light signal device emits visible light.

12. The inhalation device of claim 1 wherein the device does not include an airflow sensor.

13. An inhalation device for inhaling a vaporized substance comprising: an inlet;
an outlet;
a processor;
an atomizer positioned between the inlet and the outlet and configured to vaporize an unvaporized substance into a vaporized substance, wherein the vaporized substance flows downstream from the atomizer to the outlet via the channel;
a light signal device, wherein the light signal device emits light;
a light sensor, wherein the light sensor senses the light from the light signal device;
wherein the light signal device and the sensor are positioned in the channel such that the vaporized substance can flow past the sensor and the light signal device;
wherein the processor is configured to determine a vapor concentration using data from the sensor, wherein the processor is configured to determine an increase or decrease in vapor concentration; and
wherein the processor, using the vapor concentration and the increase or decrease in vapor concentration, is configured to meter an amount of vapor consumed by a user.

14. The inhalation device of claim 13, wherein the device is configured to produce discreet pulses of vapor at a frequency.

15. The inhalation device of claim 14, wherein the device is configured to produce vapor in the pattern of a sine wave.

16. The inhalation device of claim 13 wherein the device does not include an airflow sensor.

17. An inhalation device for inhaling a vaporized substance comprising: an inlet having an opening;
an outlet;
a processor;
an atomizer configured to vaporize an unvaporized substance into a vaporized substance;
a channel positioned between the atomizer and the outlet, wherein the vaporized substance flows downstream from the atomizer to the outlet via the channel;
a first light signal device, wherein the first light signal device emits light;
a first light sensor, wherein the light sensor senses the light from the first light signal device;
a second light signal device;
a second light sensor, wherein the second light sensor senses the light from the second light signal device;
wherein the first light signal device and first light sensor are positioned upstream of the second light signal device and the second light sensor; and
wherein the processor, using data from the first and second light sensors, is configured to meter an amount of vapor consumed by a user.

18. The inhalation device of claim 17 wherein the first light sensor and the first light signal device are positioned across from each other in the channel such that the vaporized substance can flow between the first light sensor and the first light signal device.

19. The inhalation device of claim 18 wherein the second light sensor and the second light signal device are positioned across from each other in the channel such that the vaporized substance can flow between the second light sensor and the second light signal device.

20. The inhalation device of claim 17 wherein the device does not include an airflow sensor.
